(19) United States
(12) Patent Application Publication
(10) Pub. No.: US 2013/0095190 A1
(43) Pub. Date: Apr. 18, 2013

(54) ORAL LIQUID VITAMIN AND SUPPLEMENT COMPOSITIONS

(71) Applicant: Garabet Varkes Kassabian, Tarzana, CA (US)

(72) Inventor: Garabet Varkes Kassabian, Tarzana, CA (US)

(21) Appl. No.: 13/650,421

(22) Filed: Oct. 12, 2012

Related U.S. Application Data
(60) Provisional application No. 61/546,428, filed on Oct. 12, 2011.

(51) Int. Cl. A61K 31/7056; A61P 37/04; A61K 35/64; A61K 36/45; A61K 36/889; A61P 21/00; A61P 3/06

(52) U.S. Cl. 424/537; 514/52; 424/732; 424/725

(57) ABSTRACT

Disclosed are health drink compositions. One example composition provides a health drink that includes 10-15 mg/10 ml of calcium lactate gluconate, 400-600 mcg/10 ml of folic acid, 40-50 mg/10 ml of magnesium gluconate, a combination of complex B vitamins, purified water, and natural fruit flavoring. Other example health drinks are also included in the present application.

### CROSS-REFERENCE TO RELATED APPLICATIONS

This application claims the benefit of prior filed U.S. Provisional Application Ser. No. 61/546,428, filed on Oct. 12, 2011, which is incorporated herein by reference in its entirety.

### TECHNICAL FIELD OF THE APPLICATION

This disclosure relates to a variety of vitamin and supplement compositions including health stimulating supplements and/or appetite suppressants.

### BACKGROUND OF THE APPLICATION

Those who seek to improve their current bodily health may rely on supplements, such as oral vitamins and other enhancement techniques on a frequent basis. A user's goals may include maintaining adequate vitamin levels and controlling ones appetite. Other user goals may include maintaining adequate levels of other minerals to provide optimal nourishment for the brain, joints, heart, immune system, metabolism, etc. Discovering the proper balance of vitamins and minerals to address one or more of the above-noted bodily conditions may pose a challenge for the everyday consumer. Therefore, what is needed is a variety of vitamin and supplement compositions that provide such balance.

### SUMMARY

One example embodiment provides a health drink composition that includes 10-15 mg/10 ml of calcium lactate gluconate, 400-600 mcg/10 ml of folic acid, 40-50 mg/10 ml of magnesium gluconate, a combination of complex B vitamins, purified water, and natural fruit flavoring.

Another example embodiment provides a health drink composition that includes 5000 UI-10000 UI/10 ml of vitamin A, 6-7.5 mg/10 ml of Zinc (Zinc gluconate), at least one natural berry powder or extract, purified water, and natural fruit flavoring.

Another example embodiment provides a health drink composition that includes 400-500 mg/10 ml of Acai, 100-150 mg/10 ml of CoQ10, and 650-800 mb/10 ml of Phytosterol.

### BRIEF DESCRIPTION OF THE DRAWINGS

FIG. 1A illustrates an example bodily health energy composition according to example embodiments of the present invention.

FIG. 1B illustrates an example bodily immunity composition according to example embodiments of the present invention.

FIG. 1C illustrates an example bodily heart health composition according to example embodiments of the present invention.

FIG. 1D illustrates an example bodily joint health composition according to example embodiments of the present invention.

FIG. 1E illustrates an example metabolism health composition according to example embodiments of the present invention.

FIG. 2A illustrates another example bodily health energy composition according to example embodiments of the present invention.

FIG. 2B illustrates another example bodily heart health composition according to example embodiments of the present invention.

FIG. 2C illustrates another example immunity health composition according to example embodiments of the present invention.

FIG. 2D illustrates another example bodily joint health composition according to example embodiments of the present invention.

FIG. 2E illustrates another example metabolism health composition according to example embodiments of the present invention.

FIG. 3A illustrates additional information regarding the ingredients in the bodily health energy composition according to example embodiments of the present invention.

FIG. 3B illustrates additional information regarding the ingredients in the bodily heart health composition according to example embodiments of the present invention.

FIG. 3C illustrates additional information regarding the ingredients in the immunity health composition according to example embodiments of the present invention.

FIG. 3D illustrates additional information regarding the ingredients in the bodily joint health composition according to example embodiments of the present invention.

FIG. 3E illustrates additional information regarding the ingredients in the metabolism health composition according to example embodiments of the present invention.

### DETAILED DESCRIPTION OF THE INVENTION

It will be readily understood that the components of the present invention, as generally described and illustrated in the figures herein, may be arranged and designed in a wide variety of different configurations or compositions.

The features, structures, compositions or characteristics described throughout this specification may be combined in any suitable manner in one or more embodiments. For example, the usage of the phrases “example embodiments”, “some embodiments”, or other similar language, throughout this specification refers to the fact that a particular feature, composition, or characteristic described in connection with the embodiment may be included in at least one embodiment of the present invention. Thus, appearances of the phrases “example embodiments”, “in some embodiments”, “in other embodiments”, or other similar language, throughout this specification do not necessarily all refer to the same group of embodiments, and the described features, compositions, or characteristics may be combined in any suitable manner in one or more embodiments.

One example composition according to example embodiments may include an energy booster combination of vitamins, minerals, and other ingredients. For instance, B-vitamins (B-1, B-2, B-3, B-5, B-6 and/or B-12) may be necessary for producing energy, enhancing nervous system function and assisting with other bodily functions. B-Vitamins are generally water soluble vitamins, which a human body requires. The human body should maintain a constant supply of these nutrients to function at its best. Consuming an energy booster drink daily may provide a person's body with a continuous or proper dose of these vital nutrients. One or more of the example embodiments may include certain B-vitamins or a B-complex set of vitamins, such as one or more of Thiamine (B1), Riboflavin (B2), Niacinamide (B3), Pyridoxine (B6), Pantothenic acid (B5) and Methylcobalamin (B12).

Other ingredients which may be included in the energy booster drink may include calcium, magnesium and vitamin-C. Calcium and magnesium are both important for muscle contraction and relaxation. Calcium and magnesium work together to help relax and rebuild muscles. Providing these minerals, in combination with the above-noted B-vitamins (B-Complex) in the energy booster drink can help decrease muscle cramps and soreness. Energy booster supports energy, health, vitality and an active lifestyle as well.

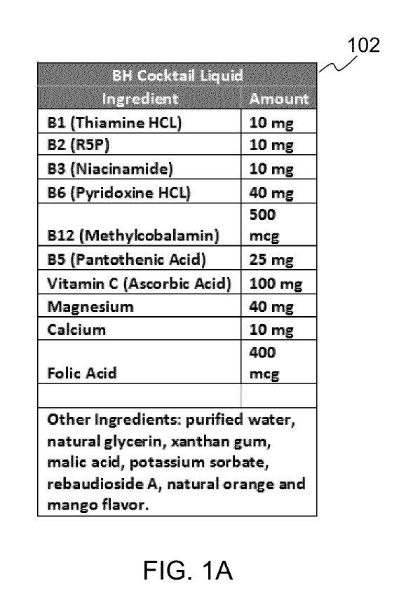

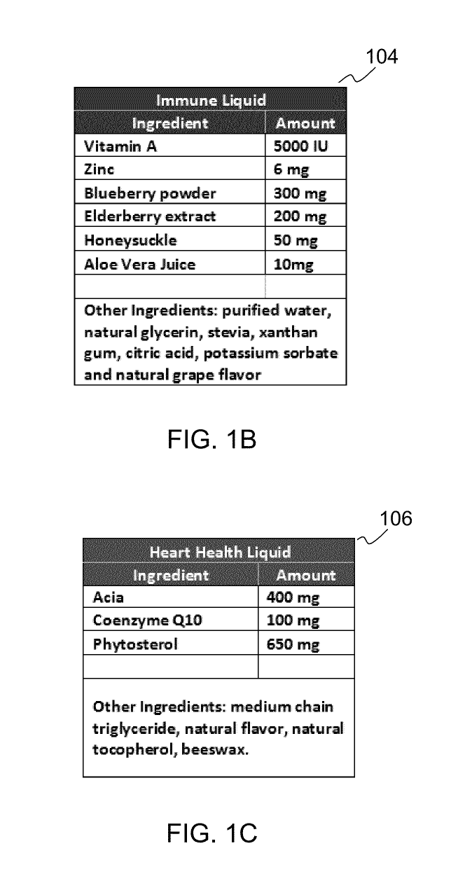

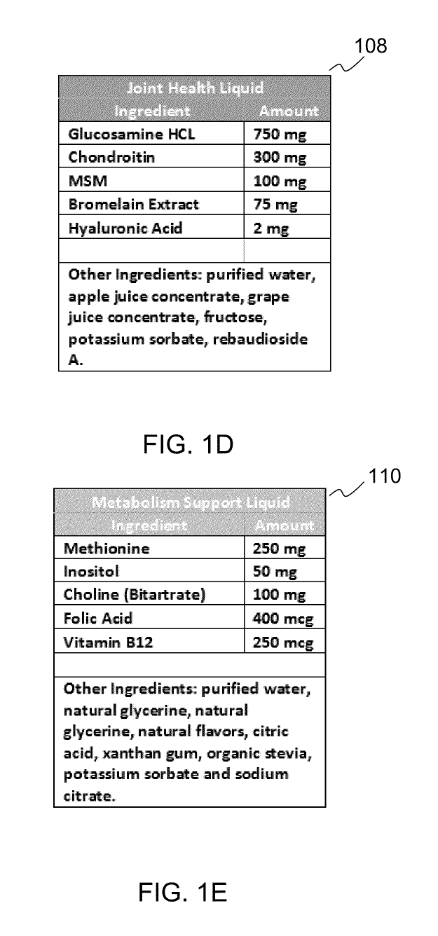

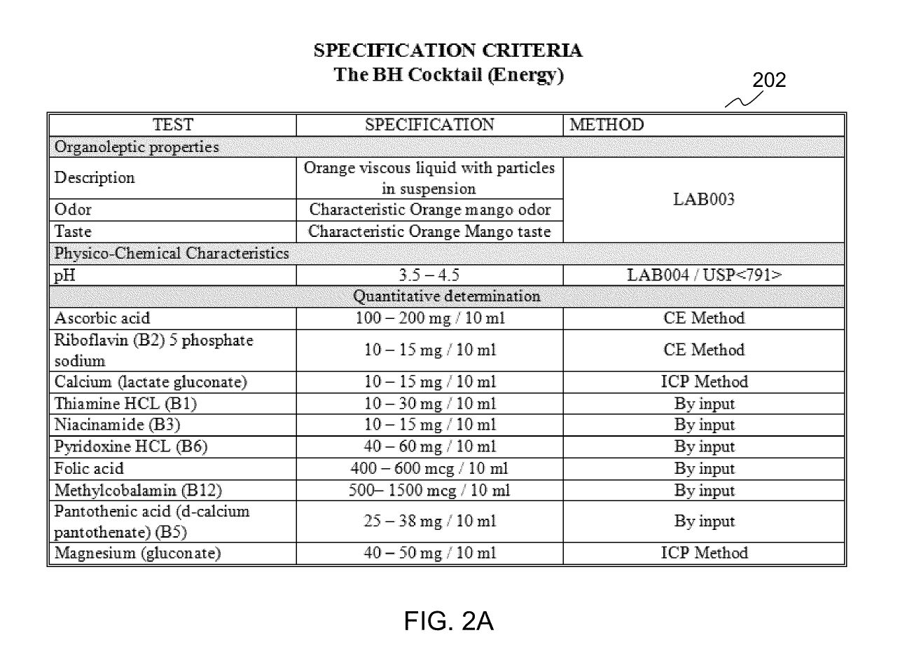

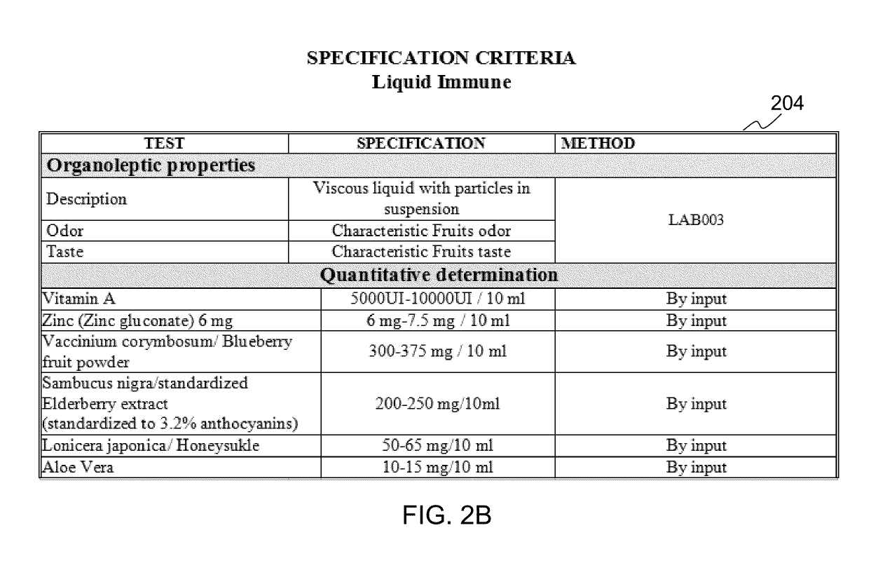

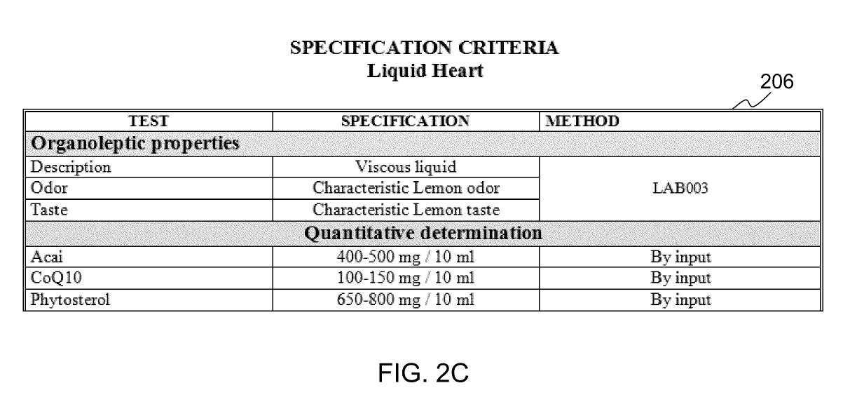

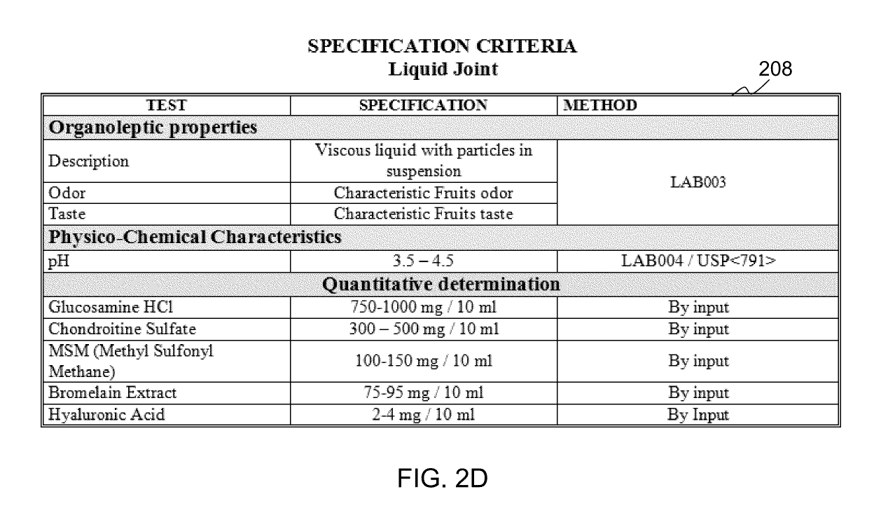

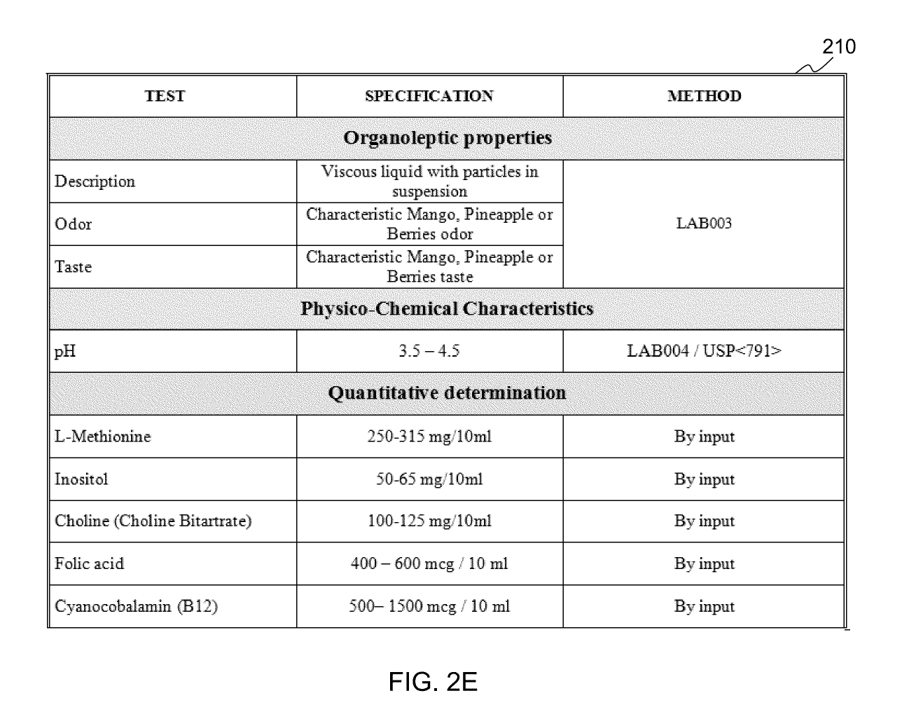

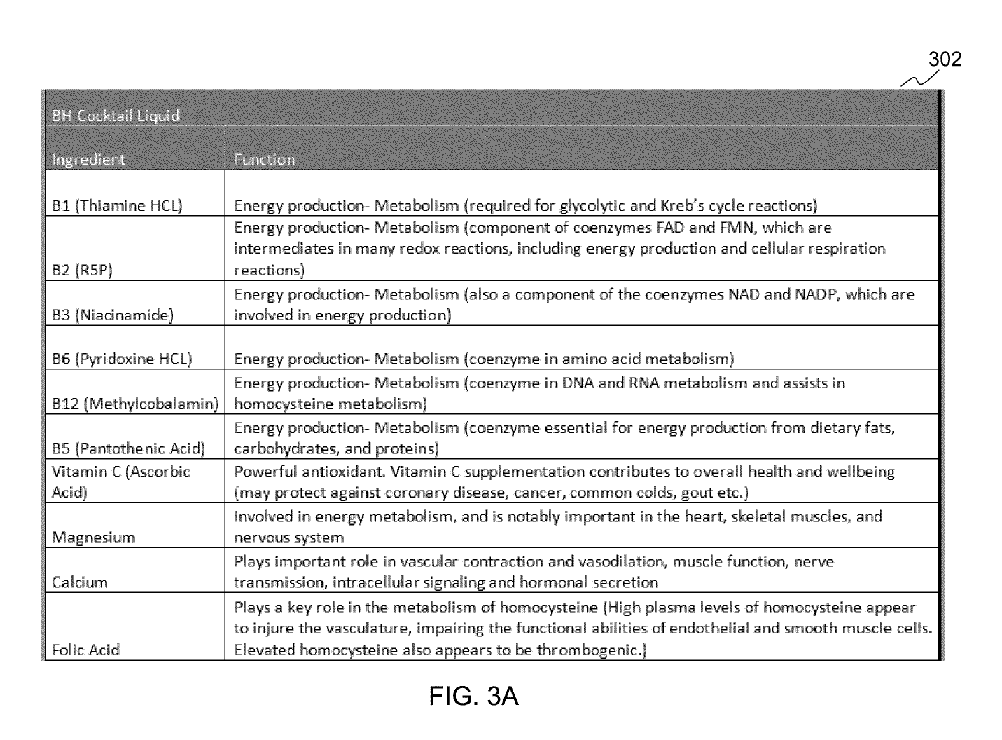

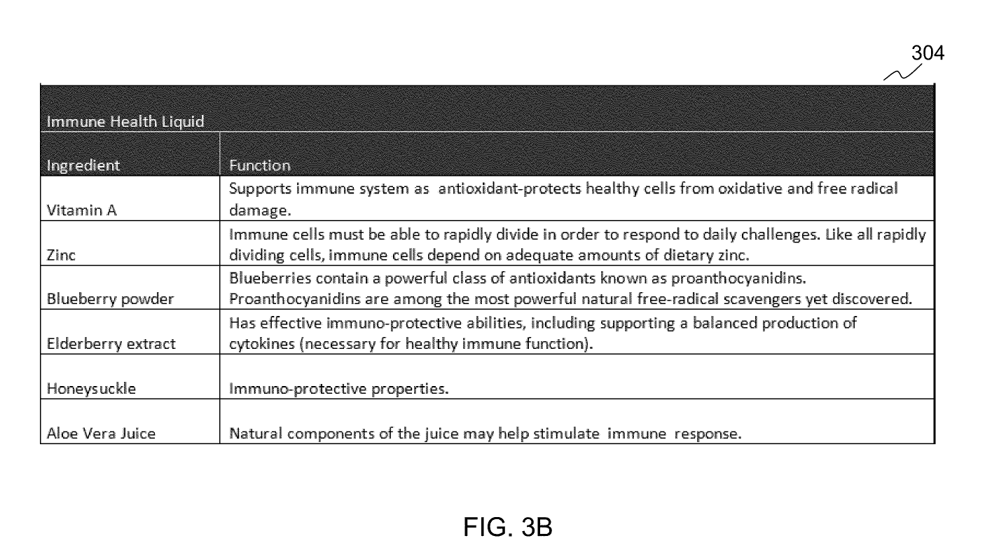

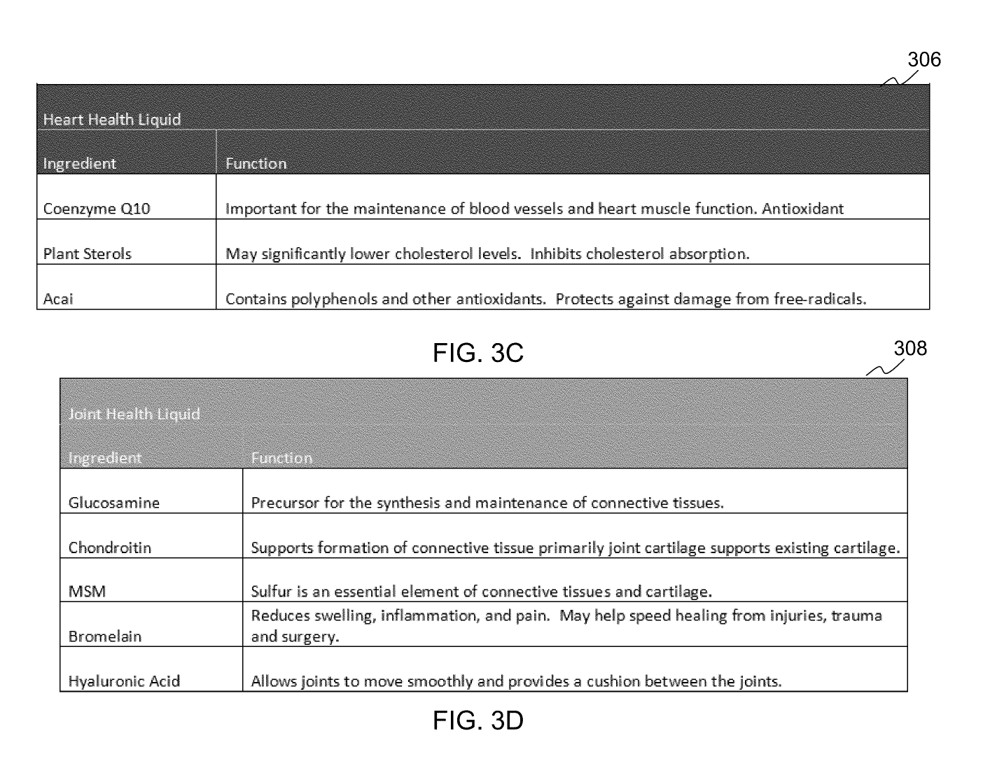

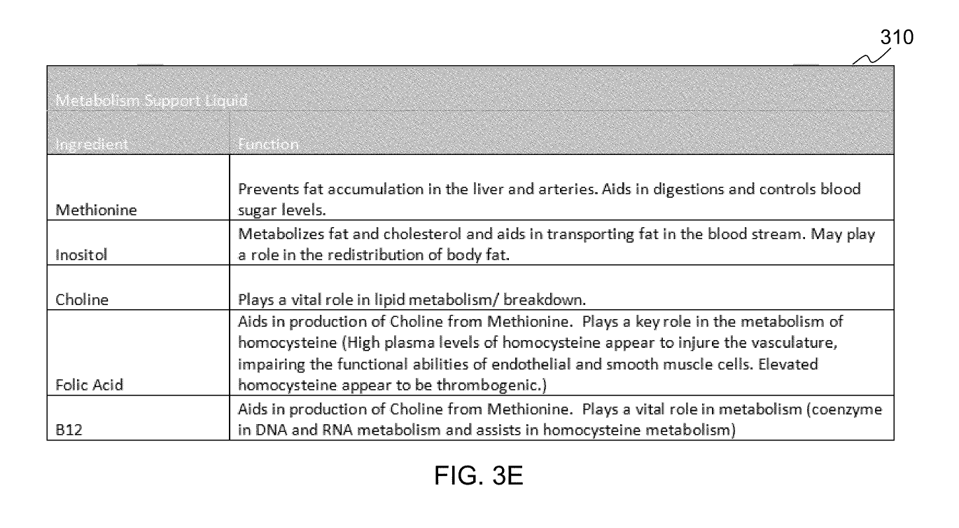

An example composition of the energy booster is illustrated in FIG. 1A. Referring to FIG. 1A, table 102 illustrates the amounts of each of the ingredients tested for an optimal energy booster supplement. Each of these ingredients may be modified from their original amounts included in table 102. For example, the energy booster drink may include different ranges of each of the specified ingredients in table 102 to accommodate slight variations in the final composition (i.e., increases or decreases to the ingredients of around 1%, 5%, 10% and 20%). Additional details of the testing, ingredients and laboratory creation methods of the energy booster health drink are illustrated in table 202 of FIG. 2A. Additional details of the ingredients in the above-noted composition are further illustrated in table 302 of FIG. 3A.

Another example composition according to example embodiments may include an immunity liquid drink combination of vitamins, minerals, and other ingredients. For instance, vitamin-A is an antioxidant that protects healthy cells from oxidative and free radical damage. Immune cells must be able to rapidly divide in order to respond to daily challenges. Dividing immune cells must have adequate amounts of dietary zinc. Blueberry powder contains proanthocyanidins which are among the most powerful natural free-radical scavengers discovered. Elderberry and honeysuckle extracts have demonstrated immuno-protective abilities, including supporting a balanced production of cytokines (necessary for healthy immune function).

An example composition of the immunity liquid drink is illustrated in FIG. 1B. Referring to FIG. 1B, table 104 illustrates the amounts of each of the ingredients tested for an optimal immunity liquid supplement. Each of these ingredients may be modified from their original amounts included in table 104. For example, the immunity liquid drink may include different ranges of each of the specified ingredients in table 104 to accommodate slight variations in the final composition (i.e., increases or decreases to the ingredients of around 1%, 5%, 10% and 20%). Additional details of the testing, ingredients and laboratory creation methods of the immunity liquid supplement drink are illustrated in table 204 of FIG. 2B. Additional details of the ingredients in the above-noted composition are further illustrated in table 304 of FIG. 3B.

Another example composition according to example embodiments may include a heart health liquid drink combination of vitamins, minerals, and other ingredients. For instance, this unique composition is placed in an medium chain triglyceride (MCT) oil base. This oil base enhances the absorption and transport of the ingredients included in the heart health liquid drink. Certain ingredients may include plant sterols, which may lower cholesterol levels and inhibit cholesterol absorption. Other ingredients may include Acai, which contains polyphenols and other antioxidants and protects against damage from free-radicals, and Coenzyme-Q10 (CoQ10), which is a potent antioxidant which plays a pivotal role in the maintenance of blood vessels and heart muscle function.

An example composition of the heart health liquid drink is illustrated in FIG. 1C. Referring to FIG. 1C, table 106 illustrates the amounts of each of the ingredients tested for an optimal heart health liquid supplement. Each of these ingredients may be modified from their original amounts included in table 106. For example, the immunity liquid drink may include different ranges of each of the specified ingredients in table 106 to accommodate slight variations in the final composition (i.e., i.e., increases or decreases to the ingredients of around 1%, 5%, 10% and 20%). Additional details of the testing, ingredients and laboratory creation methods of the heart health liquid drink are illustrated in table 206 of FIG. 2C. Additional details of the ingredients in the above-noted composition are further illustrated in table 306 of FIG. 3C.

Another example composition according to example embodiments may include a joint health liquid drink combination of vitamins, minerals, and other ingredients. For instance, one ingredient may include Glucosamine as a precursor for the synthesis and maintenance of connective tissues. Chondroitin may be used to support formation of connective tissue, primarily joint cartilage. Methyl sulfonyl methane (MSM) sulfur may be included as an essential element of connective tissues and cartilage. Bromelain may be used to reduce swelling, inflammation, and pain, which may help speed healing from injuries, trauma and surgery. Hyaluronic acid may be used to allow joints to move smoothly by providing a cushion between the joints.

An example composition of the joint health liquid drink is illustrated in FIG. 1D. Referring to FIG. 1D, table 108 illustrates the amounts of each of the ingredients tested for an optimal joint health liquid supplement. Each of these ingredients may be modified from their original amounts included in table 108. For example, the immunity liquid drink may include different ranges of each of the specified ingredients in table 108 to accommodate slight variations in the final composition (i.e., i.e., increases or decreases to the ingredients of around 1%, 5%, 10% and 20%). Additional details of the testing, ingredients and laboratory creation methods of the joint health liquid drink are illustrated in table 208 of FIG. 2D. Additional details of the ingredients in the above-noted composition are further illustrated in table 308 of FIG. 3D.

Another example composition according to example embodiments may include a metabolism control and diet control liquid drink combination of vitamins, minerals, and other ingredients. For instance, weight loss may be promoted by a special combination of lipotropic compounds in unique oral liquid for daily use. Lipotropic compounds help catalyze (speed up) the breakdown of fat. Daily dosing provides the body with a constant supply of the weapons it needs to fight fat deposits. Choline may be used to promote lipid metabolism/breakdown. Inositol is used to metabolize fat and cholesterol and aid in transporting fat in the blood stream, which may play a role in the redistribution of body fat. Methionine prevents fat accumulation in the liver and arteries and aids in digestion and controls blood sugar levels.

An example composition of the metabolism health liquid drink is illustrated in FIG. 1E. Referring to FIG. 1E, table 110 illustrates the amounts of each of the ingredients tested for an optimal metabolism health liquid supplement. Each of these ingredients may be modified from their original amounts included in table 110. For example, the metabolism liquid drink may include different ranges of each of the specified ingredients in table 110 to accommodate slight variations in the final composition (i.e., i.e., increases or decreases to the ingredients of around 1%, 5%, 10% and 20%). Additional details of the testing, ingredients and laboratory creation methods of the metabolism health liquid drink are illustrated in table 210 of FIG. 2E. Additional details of the ingredients in the above-noted composition are further illustrated in table 310 of FIG. 3E.

All of the above noted compositions may be in a liquid form which may be ingested by swallowing, or via a sublingual technique, “under the tongue” droplet insertion and absorption. In addition to liquids ingested by absorption and swallowing, the compositions noted above may also be compacted into a pill form or solid edible form (i.e., pill, gum, candy, cookie, etc.).

In order to determine the chemical compositions of the samples an inductively coupled plasma (ICP) technique may be used to measure all of the stable elements in the periodic table with the exception of gases. The ICP technique is based on atomic spectrometry. Most specifically, the ICP-ES is an emission spectrometric technique that exploits the fact that excited atoms emit energy at a given wavelength as the electrons return to their ground state. A given element emits energy at specific wavelengths peculiar to its chemical character.

Although each element emits at many wavelengths, in ICP-ES it is most common to select a single wavelength (or a very few) for a given element. The intensity of the energy emitted at that wavelength is proportional to the amount (concentration) of that element in the analyzed sample. Thus, by determining which wavelengths are present in a sample, and by determining their intensities, the analyst can quantify the composition of the given sample.

Another example procedure that may be used to identify and separate the various different ingredients of any of the above supplements may include a high performance liquid chromatography procedure (HPLC), which is a form of liquid chromatography that separates components of a mixture by using a variety of chemical interactions between the substances being analyzed. Based on the nature of the stationary phase and the separation process, four types of separation mechanisms can be specified: 1. absorption: used for the separation of isomers in a mixture, 2. ion-exchange: applied for the separation of inorganic anions/cations, low molecular weight organic acids, serums, preservatives, vitamins, sugars, ionic chelates, certain organometallic compounds and amino acids, 3. size exclusion: used for determining the tertiary and quaternary structure of purified proteins, and which is the primary technique for determining the average molecular weight of natural and synthetic polymers, and 4. chiral interaction: used to separate enantiomeric mixtures.

While preferred embodiments of the present application have been described, it is to be understood that the embodiments described are illustrative only and the scope of the application is to be defined solely by the appended claims when considered with a full range of equivalents and modifications (e.g., additional ingredients, vitamins, minerals, etc.) thereto.

What is claimed is:

1. A health drink composition comprising:
10-15 mg/10 ml of calcium lactate gluconate;
400-600 mcg/10 ml of folic acid;
40-50 mg/10 ml of magnesium gluconate;
a combination of complex B vitamins;
purified water; and
natural fruit flavoring.

2. The health drink composition of claim 1, wherein the combination of complex B vitamins include at least two of Thiamine (B1), Riboflavin (B2), Niacinamide (B3), Pyridoxine (B6), Pantothenic acid (B5) and Methylcobalamin (B12).

3. The health drink composition of claim 1, wherein the complex B vitamins consists of the group of:
10-15 mg/10 ml of Thiamine HCL (B1),
10-15 mg/10 ml of Riboflavin (B2),
10-15 mg/10 ml of Niacinamide (B3),
25-38 mg/10 ml of Pantothenic acid (B5),
40-60 mg/10 ml of Pyridoxine HCL (B6), and
500-1500 mcg/10 ml of Methylcobalamin (B12).

4. The health drink composition of claim 1, further comprising:
ascorbic acid 100-200 mg/10 ml;
glycerin;
xanthan gum;
malic acid;
potassium sorbate; and
rebaudioside A.

5. The health drink composition of claim 1, wherein the health drink pH is 3.5-4.5.

6. A health drink composition comprising:
5000 UI-10000 UI/10 ml of vitamin A;
6-7.5 mg/10 ml of Zinc (Zinc gluconate);
at least one natural berry powder or extract;
purified water; and
natural fruit flavoring.

7. The health drink composition of claim 6, wherein the at least one natural berry powder or extract comprises 300-375 mg/10 ml of Vaccinium corymbosum/blueberry fruit powder and 200-250 mg/10 ml Sambucus nigra/standardized elderberry extract.

8. The health drink composition of claim 6, wherein the health drink composition further comprises:
50-65 mg/10 ml of Lonicera japonica (Japanese Honeysuckle), and 10-15 mg/10 ml of Aloe Vera.

9. The health drink composition of claim 6, further comprising:
glycerin;
stevia
xanthan gum;
citric acid; and
potassium sorbate.

10. A health drink composition comprising:
400-500 mg/10 ml of Acai;
100-150 mg/10 ml of CoQ10; and
650-800 mb/10 ml of Phytosterol.

11. The health drink composition of claim 10, further comprising:
medium chain triglyceride;
tocopherol; and
beeswax.
